(19) United States
(12) Patent Application Publication
(10) Pub. No.: US 2025/0064317 A1
(43) Pub. Date: Feb. 27, 2025

(54) Optical Pressure Sensor for Eye with Embedded Interrogation System

(71) Applicant: Twenty Twenty Therapeutics LLC, South San Francisco, CA (US)

(72) Inventors: Supriyo Sinha, Menlo Park, CA (US); Benedict Costello, Piedmont, CA (US); Dimitri Azar, Chicago, IL (US)

(21) Appl. No.: 18/455,981

(22) Filed: Aug. 25, 2023

(51) Int. Cl. A61B 3/16; A61B 5/00

(52) U.S. Cl. CPC A61B 3/16; A61B 5/0004; A61B 5/0017; A61B 5/0084; A61B 2562/0238; A61B 2562/0247

(57) ABSTRACT

An intraocular implant comprising: a pressure sensor implantable in an eye, wherein the pressure sensor comprises a substrate having a first side coupled to a membrane to define an optical cavity with a depth that varies based on an intraocular pressure of the eye; and an enclosure coupled to a second side of the substrate to define a hermetic cavity comprising a light source and a detector operable to interrogate the pressure sensor and obtain data corresponding to an intraocular pressure of the eye based on the interrogation.

### FIELD

The subject matter of this disclosure relates to an intraocular implant having an intraocular pressure (IOP) sensor that is interrogated with a light source and a detector integrated into the implant. Such techniques are useful for treating and/or monitoring progression of eye diseases including glaucoma, but are not limited to use with the treatment of eye disease.

### BACKGROUND

Intraocular pressure (IOP) refers to the pressure of a fluid known as the aqueous humor inside the eye. The pressure is normally regulated by changes in the volume of the aqueous humor, but some individuals suffer from disorders, such as glaucoma, which cause chronic heightened IOP. Over time, heightened IOP can cause damage to the eye's optical nerve, leading to loss of vision. Presently, treatment of glaucoma mainly involves periodically administering pharmaceutical agents to the eye to decrease IOP. These drugs can be delivered, for example, by injection or eye drops. However, effective treatment of glaucoma requires adherence to dosage schedules and a knowledge of the patient's IOP. The more current or recent the measurement is, the more relevant it will be and hence the more effective the resulting treatment can be. The IOP for a given patient can vary significantly based on time of day, exercise, how recently a medication was taken, and other factors. Typically, IOP measurements are performed in a doctor's office and often no more than once or twice per year. These infrequent measurements are less able to account for variation in the patient's IOP, and may become stale due to the length of time between them. This means that any given measurement is subject to uncertainty, so it may take several IOP measurements over time to have confidence in the health of the patient's eye.

Typically, the IOP is measured using a tonometer, which is a device that is outside the eye and thus does not require a sensor within the eye. Contact tonometry is performed in a clinical setting, and the procedure requires numbing of the patient's eye, resulting in both inconvenience and discomfort. Noncontact tonometry involves directing a puff or jet of air towards the patient's eye and measuring the resulting deflection dynamics of the cornea. However, this requires a bulky and power hungry pump arrangement that may not be practical for home use, and is not as accurate as contact tonometry.

A wireless, implantable, continuous IOP monitoring system has been suggested that has a commercial pressure sensing element with digital readout, and a microelectronic chip that supports wireless power/data telemetry and a wired serial communication interface with the pressure sensing element. An on-chip integrated RF coil receives power from near-field RF coupling at 915 MHz, and transmits pressure measurement bits via RF-backscattering to an external reader. This type of system, however, may require precise optical alignment between the external reader and the sensor in order to ensure the measurement data is accurate. It may be difficult for some users holding the external reader to meet such precise alignment requirements leading to inaccurate measurements.

### SUMMARY

Measurement of IOP is critical to monitor the progression of glaucoma and to monitor effectiveness of treatments such as eye drops or surgical interventions. As previously discussed, tonometers used outside the eye are bulky and have low accuracy. It would be desirable to be able to measure IOP with a sensor that is minimally invasive inside the eye and without the need for precise alignment of an external reader. A device that can measure IOP autonomously (i.e., without the user having to perform an action to make a measurement) would be ideal as it requires lower compliance on the part of the user. An aspect of the disclosure is therefore directed to an IOP implant placed in an anterior chamber of the eye and having an IOP sensor that is interrogated with a light source and a detector integrated into the implant. In some aspects, the IOP implant, once implanted into an eye of a user, may be powered optically through ambient lighting (e.g., by sunlight, room or office lighting) rather than by a dedicated light source or by any specific action of its user.

Representatively, in some aspects, the implant may include an optical implant that may be used to measure the IOP. For example, the implant may include an IOP sensor made up of a clear glass substrate spaced apart from a clear glass membrane (e.g., by about 3-30 μm) that moves relative to the substrate depending on the IOP. On one side of the glass substrate (which might measure 70-700 microns in thickness), additional optics and electronics can be placed. For example, in one aspect, two light sources of different wavelengths could be used to interrogate the pressure sensor after reflecting through a non-polarizing beam splitter. A portion of the light reflected from the implant passes through the beam splitter and hits a photodiode detector. By cycling the light sources, a ratio of the intensities at the two wavelengths can be calculated from which the deflection of the membrane could be determined. A ratio vs IOP curve could be calibrated and allow for the IOP to be calculated via a lookup table (or equation if the function was reasonably well behaved). These optical components may be encapsulated in a hermetically sealed enclosure coupled to the substrate to protect them from the intraocular environment. Also in the encapsulation could be an application-specific integrated circuit (ASIC) and a battery. Additional electronics may further be encapsulated in the implant to optically communicate the data to a handheld reader device. For example, a transmitter and a receiver may further be integrated into the implant. The transmitter may be a light emitting diode (LED) operable to transmit the data to the reader device and the receiver may communicate with the reader device to determine when to transmit the data. An additional method would be to have a system without a battery for on-demand measurements in which the handheld reader would both provide power to the device optically and then get the readout. In this aspect, the implant would have a photovoltaic to receive the transmitted optical power from the handheld.

The implant disclosed herein may have several advantages over existing solutions. For example, the membrane side of the device, which is exposed to the aqueous humour has no electronic components and there is no need to encapsulate anything on this side. In addition, the use of an LED transmitter in the implant to communicate the data to an external reader means that the implant can be much smaller since an antenna on the order of the communication wavelength is not required. For example, discrete LEDs can be as small as 0.2 mm. Moreover, since the interrogation beam is fixed in position to the implant, the fabrication tolerances are much looser than what is required in devices in which the handheld provides the interrogation beam, which needs to work even when the beam is not perfectly aligned. Still further, the power consumption to read the implant might be considerably less than that of an electronic pressure sensor. For example, a piezoresistive sensor (which is much smaller than capacitive pressure sensors and thus more attractive for tiny pressure sensors), typically requires on the order of 10 mW during the measurement (and maybe ˜10 milliseconds to make it ˜ 0.1 mJ per measurement). On the other hand, an LED or even a vertical-cavity surface-emitting laser (VCSEL) plus a photodiode would probably consume about 1 mW of power with probably a similar time to make a measurement. Not only is the energy required to make a measurement less, the peak current draw on the battery is also smaller.

In one aspect, an intraocular implant includes a pressure sensor implantable in an eye. wherein the pressure sensor comprises a substrate having a first side coupled to a membrane to define an optical cavity with a depth that varies based on an intraocular pressure of the eye; and an enclosure coupled to a second side of the substrate to define a hermetic cavity comprising a light source and a detector operable to interrogate the pressure sensor and obtain data corresponding to an intraocular pressure of the eye based on the interrogation. In some aspects. the light source is a first light source, and the implant further includes a second light source and a non-polarizing beam splitter positioned in the hermetic cavity to interrogate the pressure sensor. In some aspects, the first light source emits a beam of light having a first wavelength and the second light sources emits a beam of light having a second wavelength through the non-polarizing beam splitter to the detector to interrogate the pressure sensor. The implant may further include an application-specific integrated circuit and a battery positioned in the hermetic cavity. In some aspects, the implant further comprises a light emitting diode in the hermetic cavity to optically transmit intraocular pressure data obtained by the interrogation of the pressure sensor to an external handheld reader device. In some aspects, the light source is arranged orthogonal to the detector. In still further aspects, the light source is a single light source having an emission spectrum that changes over time. In other aspects, a collimating lens and a beam splitter, and the light source emits a beam of light through the collimating lens to produce a number of light rays that pass through the beam splitter to interrogate different regions of the membrane. In some aspects, the detector is a first detector, and the implant further comprises a second detector, the first detector and the second detector are operable to receive a first light ray a second light ray reflected through the beam splitter, respectively. The pressure sensor may be implanted in an anterior chamber of the eye.

In other aspects, an intraocular pressure measurement system may include an intraocular implant implantable in an eye comprising a pressure sensor having a substrate having a first side coupled to a membrane to define an optical cavity with a depth that varies based on an intraocular pressure of the eye and an enclosure coupled to a second side of the substrate to define a hermetic cavity comprising a light source and a detector operable to interrogate the pressure sensor, and a transmitter operable to output data corresponding to an intraocular pressure of the eye based on the interrogation; and an external reader device comprising a processor configured to receive the output data and, based on the output, estimate the intraocular pressure of the eye. In some aspects, the light source is a first light source, and the implant further comprises a second light source and a non-polarizing beam splitter positioned in the hermetic cavity to interrogate the pressure sensor. In some aspects, the first light source emits a beam of light having a first wavelength and the second light sources emits a beam of light having a second wavelength through the non-polarizing beam splitter to the detector to interrogate the pressure sensor. In still further aspects, the implant further includes an application-specific integrated circuit and a battery positioned in the hermetic cavity. The implant further includes a light emitting diode in the hermetic cavity to optically transmit intraocular pressure data obtained by the interrogation of the pressure sensor to an external handheld reader. In some aspects, the light source is arranged orthogonal to the detector. In still further aspects, the light source is a single light source having an emission spectrum that changes over time. In other aspects, a collimating lens and a beam splitter, and the light source emits a beam of light through the collimating lens to produce a number of light rays that pass through the beam splitter to interrogate different regions of the membrane. In still further aspects, the detector is a first detector, and the implant further comprises a second detector, the first detect and the second detector are operable to receive a first light ray a second light ray reflected through the beam splitter, respectively. In other aspects, the intraocular implant is implanted in an anterior chamber of the eye.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have particular advantages not specifically recited in the above summary.

### BRIEF DESCRIPTION OF THE DRAWINGS

Several aspects of the disclosure here are illustrated by way of example and not by way of limitation in the figures of the accompanying drawings, in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1 shows an example system for measuring intraocular pressure including an IOP sensor implant implanted into an anterior chamber of an eye and an external reader device.

FIG. 2 illustrates a cross-sectional side view of one representative IOP sensor implant configuration that could be used in FIG. 1.

FIG. 3 illustrates a cross-sectional side view of one representative IOP sensor implant configuration that could be used in FIG. 1.

FIG. 4 illustrates a cross-sectional side view of one representative IOP sensor implant configuration that could be used in FIG. 1.

FIG. 5 illustrates a cross-sectional side view of one representative IOP sensor implant configuration that could be used in FIG. 1.

FIG. 6 illustrates a cross-sectional side view of one representative IOP sensor implant configuration that could be used in FIG. 1.

### DETAILED DESCRIPTION

Several aspects of the disclosure with reference to the appended drawings are now explained. Whenever the shapes, relative positions and other aspects of the parts described are not explicitly defined, the scope of the invention is not limited only to the parts shown, which are meant merely for the purpose of illustration. Also, while numerous details are set forth, it is understood that some aspects of the disclosure may be practiced without these details. In other instances, well-known circuits, structures, and techniques have not been shown in detail so as not to obscure the understanding of this description.

The terminology used herein is for the purpose of describing particular aspects only and is not intended to be limiting of the disclosure. Spatially relative terms, such as “beneath”. “below”, “lower”, “above”, “upper”, and the like may be used herein for ease of description to describe one element's or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (e.g., rotated 90 degrees or at other orientations) and the spatially relative descriptors used herein interpreted accordingly.

As used herein, the singular forms “a”, “an”, and “the” are intended to include the plural forms as well, unless the context indicates otherwise. It will be further understood that the terms “comprises” and/or “comprising” specify the presence of stated features, steps, operations, elements, and/or components, but do not preclude the presence or addition of one or more other features, steps, operations, elements, components, and/or groups thereof.

The terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

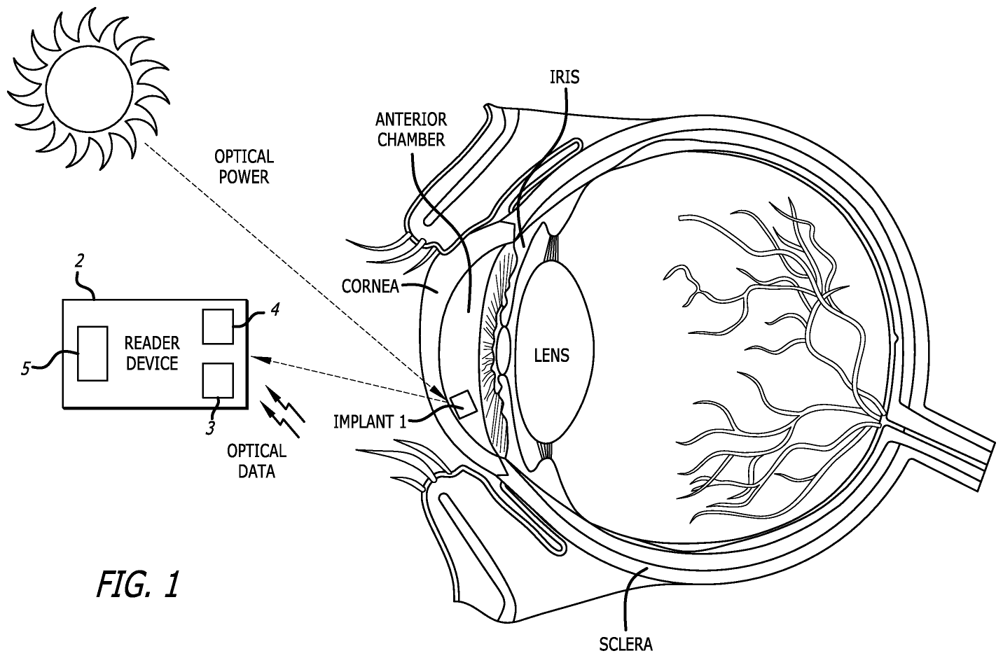

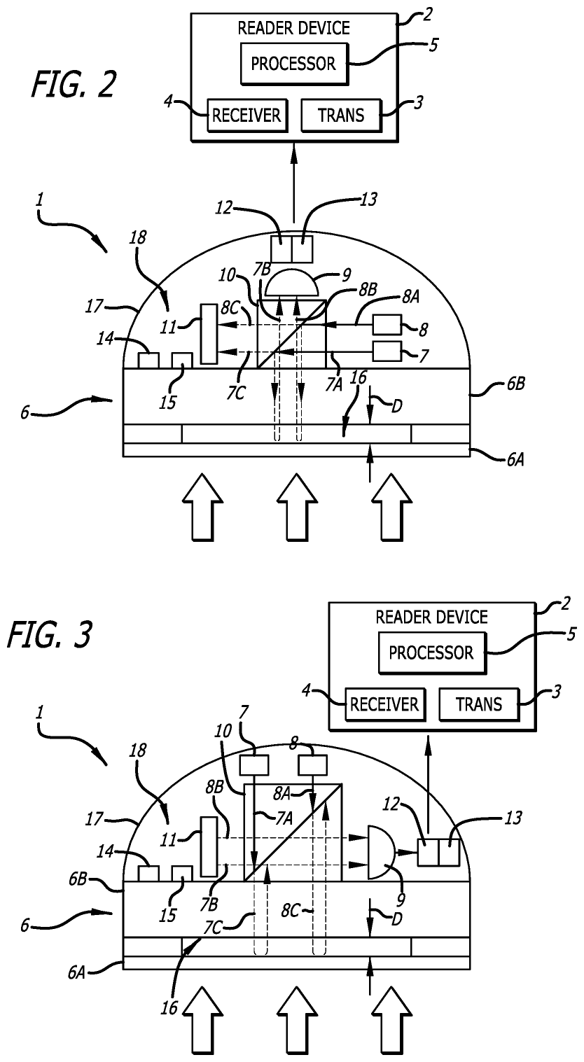

FIG. 1 illustrates an example system for measuring intraocular pressure, using an implant 1 including an IOP sensor implanted in the eye and an external reader device 2 for reading the implant 1. Implant 1 may, in some aspects, be implanted in its entirety within the anterior chamber as shown. Implant 1 may be composed of several parts or components which cooperate to enable the implant 1 to measure an IOP of the eye, as will be discussed in more detail in reference to FIG. 2. Reader device 2 may be a portable or handheld device having electronic components and/or circuitry operable to read implant 1. For example, reader device 2 may include a transmitter 3 and a receiver 4 configured to communicate with, and receive data from, implant 1. For example, receiver 4 may be configured to receive transmitted, measured pressure data from implant 1 once the reader device 2 is close to the eye, for example using one or more LEDs or other suitable photodetector arrangement. It then processes the received measured pressure data using processor 5 to inform the user about their IOP. The reader device 2 may be deemed close to the eye when it can receive the optically transmitted, measured pressure data, for example at no more than three inches away from the eye. For example, in one aspect, microelectronic circuitry of reader device 2 may transmit an optical interrogation signal that is detected by implant 1 (e.g., implant receiver). In response, implant 1 becomes aware that the reader device 2 is within range to receive the data, and therefore drives an implant transmitter (e.g., micro LED) with the measured pressure data to optically transmit the measured pressure data to reader device 2. In addition, bi-directional communication between the device receiver/transmitter and the implant receiver/transmitter may help further ensure implant 1 is aware of the fact that the measured pressure data has been uploaded into the reader device 2. In a further aspect, implant 1 may have sufficient data storage capacity to store or log all measurements that are taken between measured pressure data upload times.

Referring now in more detail to the implant configuration, FIG. 2 illustrates a cross-sectional side view of the various components of implant of FIG. 1. Implant 1 may include an intraocular pressure (IOP) sensor 6 including a rigid substrate 6A and a flexible membrane 6B as previously discussed. Substrate 6A and membrane 6B may be spaced from one another to form an optical cavity 16 with a depth (D) that varies based on an intraocular pressure of the eye. For example, the production and/or outflow of the aqueous humour within the anterior chamber influences the IOP. Since implant 1 is implanted within the anterior chamber, changes in the IOP will apply a pressure to membrane 6A, for example as illustrated by the arrows, which in turn, changes the depth (D) of optical cavity 16. Membrane 6A bends predictably and reversibly in proportion to increasing IOP, which in turn reduces the distance (or depth (D)) between the centers (or other corresponding points) of the surfaces of substrate 6B and membrane 6A. For example, the distance or depth (D) between a point on the surface of membrane 6A and a point on a surface of the substrate 6B decreases as pressure increases. Membrane 6A and substrate 6B differ in stiffness, such that for a range of expected IOP, the substrate exhibits little-to-no deflection, whereas the membrane exhibits measurable deflection. In operation, membrane 6A exhibits flexibility with respect to IOP, whereas the substrate exhibits rigidity. These changes in depth (D) of cavity 16 due to the bending or deflection of membrane 6A in response to IOP pressure changes may be interrogated and detected by the optical and electrical components encapsulated on the other side of substrate 6A by enclosure 17.

Representatively, enclosure 17 may form a hermetically sealed cavity 18 along the other side of substrate 6A that is protected from the intraocular environment and contains optical and electrical components for interrogating IOP sensor 6. In some aspects, such components may include light sources 7 and 8 of different wavelengths that can be used to interrogate IOP sensor 6 after reflecting through a non-polarizing beam splitter 10. Representatively, beam splitter 10 may be a non-polarizing cube beam splitter positioned along a top side of substrate 6B as shown. Light sources 7 and 8 may also be positioned along the top side of substrate 6B and arranged relative to beam splitter 10 so that they emit incident beams of light 7A and 8A through a side face of beam splitter 10. Portions of the incident beams of light 7A, 8A are then reflected through the beam splitter 10 to interrogate sensor 6 as illustrated by the dashed lines 7B, 8B. Representatively, to interrogate sensor 6, beam splitter 10 reflects beams of light 7B, 8B from each light source downward toward membrane 6A and substrate 6B of sensor 6. Beams of light 7B, 8B are then reflected off surfaces of membrane 6A and/or substrate 6B at different intensities depending on the depth (D) of cavity 16 back through beam splitter 10 to detector 9 as shown. In this aspect, by cycling the light sources, a ratio of the intensities can be calculated from which a deflection of membrane 6A may be determined. A ratio of intensities vs IOP curve could further be calibrated and allow for the IOP to be calculated via a lookup table (or equation if the function is reasonably well behaved). Detector 9 may, in some aspects, be a photodiode that is arranged along a top face of beam splitter 10. In this aspect, detector 9 and light sources 7, 8 may be considered to be arranged orthogonal to one another. Light sources 7, 8 could be light emitting diodes (LED) or vertical-cavity surface-emitting lasers (VCSEL) that emit light beams of different wavelengths. In still further aspects, it is further contemplated that in some aspects, the spectrum of the light source may change sufficiently over time (for example during warmup or at two different current levels) such that a single light source and detector could be used to make the measurement. In addition, it is further contemplated that where LEDs with sufficient spectral bandwidth are used, a dichroic beam splitter could be used to split the signal and ratiometric detection could be used with a single source.

An LED transmitter 12 and a receiver 13 may further be encapsulated within enclosure 17 on the top side of substrate 6B. Representatively, LED transmitter 12 may be arranged above detector 9 as shown. LED transmitter 12 may be configured to receive the intraocular pressure data or signal from detector 9 and output or transmit the data to the external reader device 2. Receiver 13 may further be arranged within enclosure 17 and may be configured to receive a signal from external reader device 2 that lets implant 1 know to transmit the data to device 2 (e.g., that device 2 is within range and/or the device receiver 4 is otherwise ready to receive data from the implant). In other aspects, LED transmitter 12 and receiver 13 could be omitted and instead light sources 7, 8 and detector 9 used for interrogation of sensor 6 could be configured such that they can also be used for data communication (e.g., handshaking and data transmission) with reader device 2.

In still further aspects, an application-specific integrated circuit (ASIC) 14 and battery 15 are also encapsulated within the hermetic cavity 18 formed by enclosure 17 as shown. ASIC 14 may be used to, for example, drive light sources 7, 8 for interrogation of sensor 6 and/or transmission of data by LED transmitter 12. In some aspects, ASIC 14 may have a watchdog timer that triggers the taking of measurements of the IOP sensor 6 (or producing measured pressure data) at intervals programmed into the watchdog timer. These intervals may be regular (e.g., one measurement every fifteen minutes) or they may be irregular (e.g., one measurement every half hour, and once a day a burst of measurements is taken every five hundred milliseconds for ten seconds.) Each measurement may be stored in memory associated with ASIC 14 (e.g., in a static random access memory module) along with a timestamp for the measurement, until it is time to transmit the measurement (as produced measured pressure data) to the external reader device 2.

Battery 15 may be used to store energy to power the various electronic components and/or devices within implant 1. For example, battery 15 may be used to provide enough energy for taking the previously discussed measurements even when implant 1 is not being actively optically powered by ambient light. For example, when the user's eye (in which implant 1 has been implanted) has not been exposed to ambient light to power implant 1 or recharge the battery 15 (e.g., solar light outside), battery 15 may store enough energy to power the taking of measurements for a reasonable amount of time. Note, the term “battery” is used generically here, to refer to any type of rechargeable solid state device (e.g., a solid state battery) or other electricity storage device that can be charged and/or recharged.

In addition, in some aspects, although not shown, implant 1 may further include a photovoltaic element configured to convert incident ambient light, e.g., solar, into energy that it supplies to operate the implant components as previously discussed. The photovoltaic element may be configured to continuously provide its converted energy to be stored temporarily within, or recharge, battery 15.

In still further aspects, a beam dump 11 may be arranged along a face of the beam splitter 10 opposite light sources 7, 8. Beam dump 11 may be configured to absorb the portions of beams of light 7A, 8A transmitted through beam splitter 10. Representatively, as illustrated in FIG. 2, light sources 7, 8 emit incident beams of light 7A, 8A through the side face of beam splitter 10. These incident beams of light 7A, 8A are then split by beam splitter 10 into reflected beams 7B, 8B and transmitted beams 7C, 8C. Reflected beams 7B, 8B are reflected toward, and interrogate, sensor 6 as previously discussed. Transmitted beams 7C, 8C continue through beam splitter 10 and are absorbed by beam dump 11.

FIG. 3 illustrates a cross-sectional side view of another representative arrangement of the various components of implant 1 discussed in reference to FIG. 1. Implant 1 shown in FIG. 3 may have all the same components as the implant described in reference to FIG. 2. Thus, a detailed description of each of the same components described in reference to FIG. 2 is omitted for the sake of conciseness. In the implant configuration of FIG. 3, however, the position of light sources 7, 8 and detector 9 are interchanged such that beams 7C, 8C transmitted from beam splitter 10 interrogate sensor 6 and detector 9 is placed in the reflected path. Representatively, in this configuration, light sources 7, 8 are arranged along the top face of beam splitter 10 and detector 9 is arranged along the side face of beam splitter 10. In this aspect, light sources 7, 8 and detector 9 are still arranged orthogonal to one another, but detector is now arranged along the side face of the beam splitter 10 opposite beam dump 11. To interrogate sensor 6 in this configuration, light sources 7, 8, emit incident light beams 7A, 8A through the top face of beam splitter 10. Light beams 7A, 8A are split by beam splitter 10 into reflected light beams 7B, 8B that are reflected toward beam dump 11, and transmitted light beams 7C, 7B that continue through beam splitter 10 to interrogate sensor 6 as previously discussed. Representatively, transmitted light beams 7C, 7B reflect off of sensor 6 (e.g., membrane 6A and/or substrate 6B) at different intensities back toward beam splitter 10, where they are then reflected toward detector 9. Detector 9, in turn, communicates the corresponding intraocular pressure data to LED transmitter 12, which then transmits or outputs the data to reader device 2 where it is processed by processor 5 to determine IOP as previously discussed.

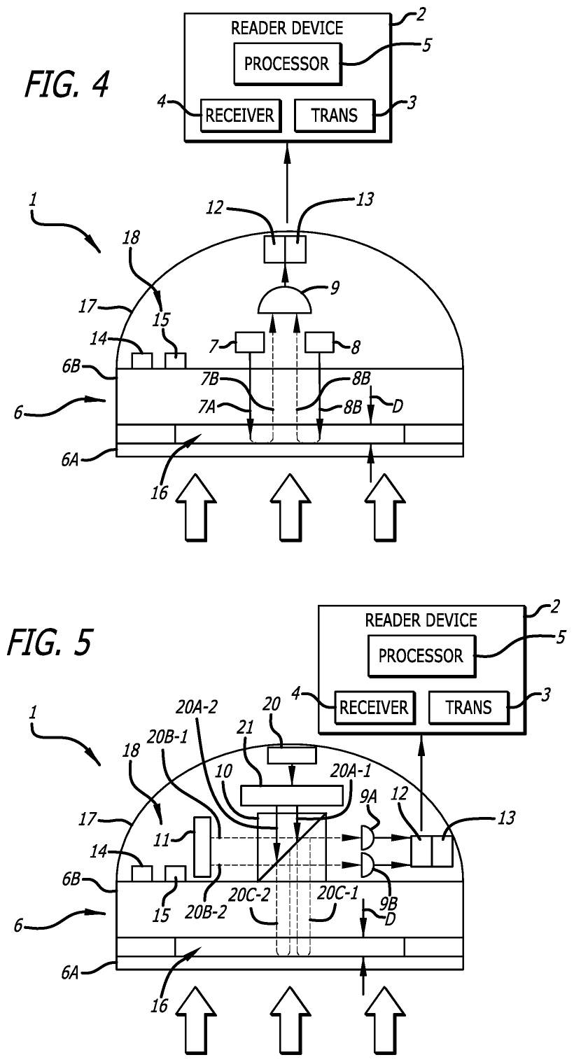

FIG. 4 illustrates a cross-sectional side view of another representative arrangement of the various components of implant 1 discussed in reference to FIG. 1. Implant 1 shown in FIG. 4 may have some of the same components as the implant described in reference to FIG. 2. Thus, a detailed description of each of the same components described in reference to FIG. 2 is omitted for the sake of conciseness. In the implant configuration of FIG. 4, however, detector 9 is aligned with (e.g., directly above) light sources 7, 8 such that a beam splitter is not necessary. In this aspect, the previously discussed beam splitter 10 is omitted. To interrogate sensor 6, light sources 7, 8 are positioned such that they emit incident beams of light 7A, 8A in a direction opposite the detector 9 (e.g., downward) toward sensor 6. Beams of light 7A, 8A then reflect off of membrane 6A and/or substrate 6B at different intensities and the reflected beams of light 7B, 8B pass back through the implant directly to detector 9. Detector 9, in turn, communicates the corresponding intraocular pressure data to LED transmitter 12, which then transmits or outputs the data to reader device 2 where it is processed by processor 5 to determine IOP as previously discussed.

FIG. 5 illustrates a cross-sectional side view of another representative arrangement of the various components of implant 1 discussed in reference to FIG. 1. Implant 1 shown in FIG. 5 may have most of the same components as the implant described in reference to FIG. 2. Thus, a detailed description of each of the same or duplicate components described in reference to FIG. 2 is omitted for the sake of conciseness. In the implant configuration of FIG. 5, however, a single collimated light source 20 and two detectors 9A, 9B are used to interrogate sensor 6. In this aspect, multiple light rays are created from a single light source 20 that can interrogate multiple regions of sensor 6. For example, one light ray may interrogate a center of membrane 6A while another light ray interrogates an off-center region of membrane 6A. Representatively, in this configuration, light source 20 is similar to the previously discussed light sources, however, a collimating lens 21 is positioned between light source 20 and beam splitter 10. The collimating lens 21 may be any type of lens operable to collimate the light beam emitted by light source 20 into multiple parallel light rays or beams 20A-1 and 20A-2 as shown. Representatively, in some aspects, lens 21 may be a thin lens such as a diffractive optic lens placed right after light source 20 to collimate the beam. Light rays or beams 20A-1 and 20A-2 then pass through the top face of beam splitter 10. Beam splitter 10 then splits beams 20A-1 and 20A-2 into reflected beams 20B-1 and 20B-2 that are reflected toward beam dump 11 and transmitted beams 20C-1 and 20C-2 that are transmitted to sensor 6. The transmitted beams 20C-1 and 20C-2 reflect off of membrane 6A and/or substrate 6B at different intensities back toward beam splitter 10. Each of transmitted beams 20C-1 and 20C-2 are then reflected by beam splitter 10 back to detectors 9A, 9B, respectively. Detectors 9A, 9B, in turn, communicates the corresponding intraocular pressure data to LED transmitter 12, which then transmits or otherwise outputs the data to reader device 2 where it is processed by processor 5 to determine IOP as previously discussed. In addition, it is further contemplated that in some aspects, where light source 20 is a VCSEL, a polarizing beam splitter (PBS) could be used with a quarter-wave plate (QWP) to allow for more efficient use of the signal. Alternatively, with an LED light source, the same thing could be done if a polarizer was also placed after the LED light source.

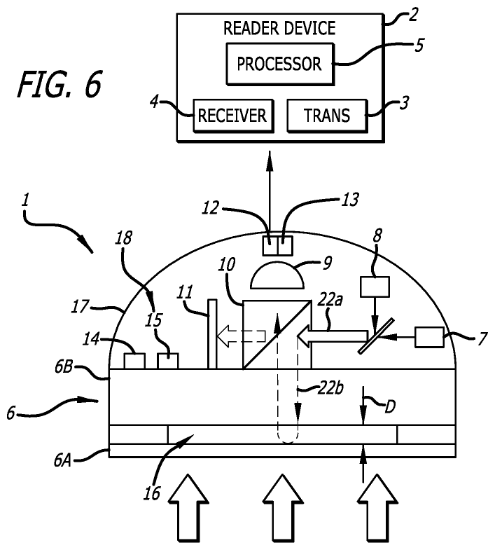

FIG. 6 illustrates a cross-sectional side view of another representative arrangement of the various components of implant 1 discussed in reference to FIG. 1. Implant 1 shown in FIG. 6 may have all the same components as the implant described in reference to FIG. 2. Thus, a detailed description of each of the same components described in reference to FIG. 2 is omitted for the sake of conciseness. In the implant configuration of FIG. 6, however, dichroic mirror 24 is further included after light sources 7, 8 to spectrally combine the emitted incident light beams from light sources 7, 8 into a completely collinear light beam 22A. For example, dichroic mirror 24 may be positioned between light sources 7, 8 and beam splitter 10 as shown. Dichroic mirror 24 may be arranged to spectrally combine the light beams from light sources 7, 8 into collinear light beam 22A, which then passes through the side face of beam splitter 10. Collinear light beam 22A may then be split by beam splitter 10 into reflected light beam 22B that is reflected toward sensor 6 to interrogate sensor 6 as previously discussed. Representatively, collinear light beam 22A reflects off of sensor 6 (e.g., membrane 6A and/or substrate 6B) back through beam splitter 10 to detector 9. Detector 9, in turn, communicates the corresponding intraocular pressure data to LED transmitter 12, which then transmits or outputs the data to reader device 2 where it is processed by processor 5 to determine IOP as previously discussed.

While certain aspects have been described and shown in the accompanying drawings, it is to be understood that such are merely illustrative of and not restrictive on the broad invention, and that the invention is not limited to the specific constructions and arrangements shown and described, since various other modifications may occur to those of ordinary skill in the art. For example, while implant 1 is shown positioned in the anterior chamber, it is contemplated that in some configurations, implant could be implanted in the cornea or sclera. In addition, regarding the reader device 2, different types of photo-emitters and photodetectors may be used in the transmitter and the receiver. Also, while FIG. 1 shows the Sun as providing its sunlight to power implant 1, other sources of optical power are possible such as artificial room lighting or a lamp that is within an accessory worn by the user (e.g., eyeglasses). The description is thus to be regarded as illustrative instead of limiting.

What is claimed is:

1. An intraocular implant comprising:
a pressure sensor implantable in an eye, wherein the pressure sensor comprises a substrate having a first side coupled to a membrane to define an optical cavity with a depth that varies based on an intraocular pressure of the eye; and
an enclosure coupled to a second side of the substrate to define a hermetic cavity comprising a light source and a detector operable to interrogate the pressure sensor and obtain data corresponding to an intraocular pressure of the eye based on the interrogation.

2. The intraocular implant of claim 1 wherein the light source is a first light source, and the implant further comprises a second light source and a non-polarizing beam splitter positioned in the hermetic cavity to interrogate the pressure sensor.

3. The intraocular implant of claim 2 wherein the first light source emits a beam of light having a first wavelength and the second light sources emits a beam of light having a second wavelength through the non-polarizing beam splitter to the detector to interrogate the pressure sensor.

4. The intraocular implant of claim 1 wherein the implant further comprises an application-specific integrated circuit and a battery positioned in the hermetic cavity.

5. The intraocular implant of claim 1 wherein the implant further comprises a light emitting diode in the hermetic cavity to optically transmit intraocular pressure data obtained by the interrogation of the pressure sensor to an external handheld reader device.

6. The intraocular implant of claim 1 wherein the light source is arranged orthogonal to the detector.

7. The intraocular implant of claim 1 wherein the light source is a single light source having an emission spectrum that changes over time.

8. The intraocular implant of claim 1 further comprising a collimating lens and a beam splitter, and the light source emits a beam of light through the collimating lens to produce a number of light rays that pass through the beam splitter to interrogate different regions of the membrane.

9. The intraocular implant of claim 8 wherein the detector is a first detector, and the implant further comprises a second detector, the first detect and the second detector are operable to receive a first light ray a second light ray reflected through the beam splitter, respectively.

10. The intraocular implant of claim 1 wherein the pressure sensor is implanted in an anterior chamber of the eye.

11. An intraocular pressure measurement system, comprising:
an intraocular implant implantable in an eye comprising a pressure sensor having a substrate having a first side coupled to a membrane to define an optical cavity with a depth that varies based on an intraocular pressure of the eye and an enclosure coupled to a second side of the substrate to define a hermetic cavity comprising a light source and a detector operable to interrogate the pressure sensor, and a transmitter operable to output data corresponding to an intraocular pressure of the eye based on the interrogation; and
an external reader device comprising a processor configured to receive the output data and, based on the output, estimate the intraocular pressure of the eye.

12. The system of claim 11 wherein the light source is a first light source, and the implant further comprises a second light source and a non-polarizing beam splitter positioned in the hermetic cavity to interrogate the pressure sensor.

13. The system of claim 12 wherein the first light source emits a beam of light having a first wavelength and the second light source emits a beam of light having a second wavelength through the non-polarizing beam splitter to the detector to interrogate the pressure sensor.

14. The system of claim 11 wherein the implant further comprises an application-specific integrated circuit and a battery positioned in the hermetic cavity.

15. The system of claim 11 wherein the implant further comprises a light emitting diode in the hermetic cavity to optically transmit intraocular pressure data obtained by the interrogation of the pressure sensor to an external handheld reader.

16. The system of claim 11 wherein the light source is arranged orthogonal to the detector.

17. The system of claim 11 wherein the light source is a single light source having an emission spectrum that changes over time.

18. The system of claim 11 further comprising a collimating lens and a beam splitter, and the light source emits a beam of light through the collimating lens to produce a number of light rays that pass through the beam splitter to interrogate different regions of the membrane.

19. The system of claim 18 wherein the detector is a first detector, and the implant further comprises a second detector, the first detect and the second detector are operable to receive a first light ray a second light ray reflected through the beam splitter, respectively.

20. The system of claim 11 wherein the intraocular implant is implanted in an anterior chamber of the eye.
